(19) United States
(12) Patent Application Publication
(10) Pub. No.: US 2009/0138071 A1
(43) Pub. Date: May 28, 2009

(54) Cardia Stent

(76) Inventors: Yingsheng Cheng, Shanghai (CN); Xing Zhou, Guangdong (CN); Ming Zhu, Beijing (CN)

Correspondence Address: Myers Andras Sherman LLP, 19900 MacArthur Blvd., Suite 1150, Irvine, CA, 92612, US

(21) Appl. No.: 12/084,470

(22) PCT Filed: Nov. 16, 2006
(86) PCT No.: PCT/CN2006/003078

§ 371 Date: May 1, 2008

(30) Foreign Application Priority Data
Nov. 16, 2005 (CN) 200510101177.1

(51) Int. Cl. A61F 2/06

(52) U.S. Cl. 623/1.15; 623/1.51

(57) ABSTRACT

The present invention relates to a medical stent, and in particular to a cardia stent for treatment on the narrow carida of the oesophagus. The cardia stent according to the present invention is woven by wires of shape memory NiTi alloy. There is a drum-shaped locating port at its upper end, and a trumpet-shaped locating port at its lower end. The upper drum-shaped locating port is connected with the lower trumpet-shaped locating port by a supporting net tube. The remaining portions other than the drum-shaped locating port are coated with a membrane of medical flexible material that can be implanted into a human body. The cardia stent comprises at least an anti-reflux valve. The anti-reflux valve is of a triple-petal structure protruding downward that is made from a membrane of medical flexible material that can be implanted into a human body. The cardia stent has the advantages of: difficult to shift, matching with carida anatomy, high anti-reflux ability, easy operation, resisting the corrosion of gastric juice, and it can be used in expansion treatment on the narrow carida caused by variable reasons.

### FIELD OF THE INVENTION

The present invention relates to a medical stent, and in particular to a cardia stent for expansion treatment on the narrow carida of the oesophagus.

### BACKGROUND OF THE INVENTION

In the prior art, the narrow carida is caused by variable reasons, for example, cardiac cancer, cardiac disorder, esophageal hiatal hernia, and other physical or chemistry damage, and will be caused by surgery treatment. Ming ZHU etc. had disclosed an intra-cavity support for medical in Chinese patent application No. 97200206.5 published on Jun. 10, 1998 (CN2283469Y). But because the shape and position of the carida, it needs to develop a carida stent which could be suitable for the expansion treatment on the narrow carida caused by variable reasons.

### SUMMARY OF THE INVENTION

The object of the present invention is to provide a cardia stent for expansion treatment on the narrow carida. The cardia stent has the advantages of: difficult to shift, matching with carida anatomy, high anti-reflux ability, easy operation, resisting the corrosion of gastric juice, and it can be used in expansion treatment on the narrow carida caused by variable reasons.

A cardia stent according to the present invention is characterized in:

a. said cardia stent is woven by wires of shape memory NiTi alloy;

b. a drum-shaped locating port, disposed at the upper end of said cardia stent;

c. a trumpet-shaped locating port, disposed at the lower end of said cardia stent;

d. a supporting net tube having a ability of resisting constringency of the cardia, connected with said upper drum-shaped locating port and said lower trumpet-shaped locating port;

e. the remaining portions other than said drum-shaped locating port are coated with a membrane of medical flexible material that can be implanted into a human body; and

f. as least one anti-reflux valve, disposed at the connection part between said lower trumpet-shaped locating port and said supporting net tube, or disposed at the connection part between said upper drum-shaped locating port and said supporting net tube, or disposed at the inner part of said supporting net tube, wherein said anti-reflux valve is of a triple-petal structure protruding downward that is made from a membrane of medical flexible material that can be implanted into a human body.

According to the cardia stent of the present invention, the medical flexible material that can be implanted into a human body are selected from the following materials: silicon rubber, Polyurethane, Polytetrafluoroethylene, and Polypropylene.

Preferred cardia stent of the present invention comprises two anti-reflux valves, and the vertical projection between the split of three petals of first anti-reflux valve and the split of three petals of second anti-reflux valve is not overlap. That forms a cardia stent with a double-layer non-overlap anti-reflux device which can effectively increase the anti-reflux ability with better ventilation.

In a preferred embodiment, the cardia stent further comprising a retrieval string, installed in said upper drum-shaped locating port, which enables said upper drum-shaped locating port to be constringed. The retrieval string is used to regulate the position of the cardia stent and take the whole cardia stent out of the human body easily according the clinical situation.

The cardia carrier of the present invention utilizes a supporting net tube to match the upper locating port and the lower locating port, which has better positioning function and is difficult to shift. Wherein, the lower trumpet-shaped locating port and the supporting net tube are coated with a medical flexible material membrane to increase the ability of resisting the corrosion of gastric juice when the cardia carrier is inserted into the gastric juice. And a double-layer non-overlap anti-reflux device can effectively increase the anti-reflux ability with better ventilation. Further, a retrieval string is used to regulate the position of the cardia stent and take the whole cardia stent out of the human body easily according the clinical situation. The cardia stent has the advantages of: difficult to shift, matching with carida anatomy, high anti-reflux ability, easy operation, resisting the corrosion of gastric juice, and it can be used in expansion treatment on the narrow carida caused by variable reasons.

### BRIEF DESCRIPTION OF THE DRAWINGS

The invention is herein described, by way of example only, with reference to the accompanying drawings, wherein:

FIG. 1 is a perspective view illustrating the structure of a cardia stent with one anti-reflux valve according to the present invention;

FIG. 2 is a tridimensional graph illustrating the structure of the cardia stent of FIG. 1;

FIG. 3 is a perspective view illustrating the structure of a cardia stent with two anti-reflux valves according to the present invention;

FIG. 4 is a tridimensional graph illustrating the structure of the cardia stent of FIG. 3;

FIG. 5 is a perspective view illustrating a vertical projection between the split of three petals of first anti-reflux valve and the split of three petals of second anti-reflux valve;

FIG. 6 is a perspective view illustrating the structure of a cardia stent with one anti-reflux valve and a retrieval string according to the present invention;

FIG. 7 is a tridimensional graph illustrating the structure of the cardia stent of FIG. 6;

FIG. 8 is a perspective view illustrating the structure of a cardia stent with two anti-reflux valves and a retrieval string according to the present invention;

FIG. 9 is a tridimensional graph illustrating the structure of the cardia stent of FIG. 8; and

FIG. 10 is a perspective view illustrating the mechanism of the cardia stent according to the present invention.

The following list is a legend of the numbering of the application illustrations:
1 drum-shaped locating port
2 trumpet-shaped locating port
3 supporting net tube
4 membrane of medical flexible material that can be implanted into a human body
5 anti-reflux valve
6 retrieval string
7 split of three petals of the anti-reflux valve
8 wires of shape memory NiTi alloy
9 oesophagus
10 stomach
11 cardia stent according to the present invention

### DETAILED DESCRIPTION OF THE PREFERRED EMBODIMENT

### Example 1

### A Cardia Stent with One Anti-Reflux Valve

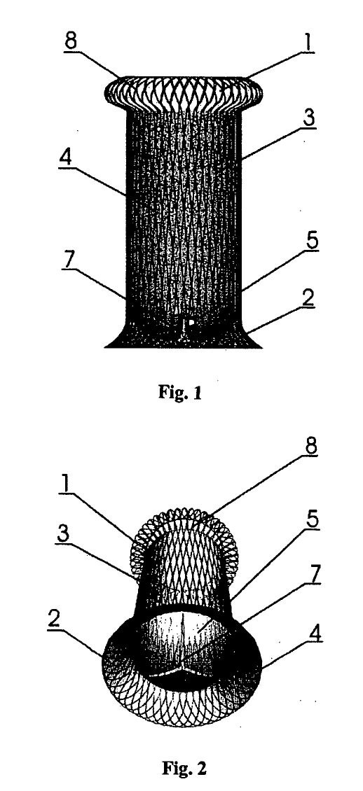

In this example, as illustrated in FIG. 1 and FIG. 2, a cardia stent is woven by wires 8 of shape memory NiTi alloy, said shape memory alloy has a recovery temperature between 25° C. and 33° C. and has a diameter between 0.2 mm and 0.25 mm. The cardia stent is finalized the design by heat treatment, and the finalized cardia stent comprises: a drum-shaped locating port 1, disposed at the upper end of said cardia stent; a trumpet-shaped locating port 2, disposed at the lower end of said cardia stent; and a supporting net tube 3 having a ability of resisting constringency of the cardia, connected with the upper drum-shaped locating port 1 and the lower trumpet-shaped locating port 2. The remaining portions other than said drum-shaped locating port 1 are coated with a membrane 4 of medical flexible material (for example, silicon rubber). We make a semi-spherical membrane of medical flexible material (for example, silicon rubber) between the lower trumpet-shaped locating port 2 and said supporting net tube 3, then shear the semi-spherical membrane into three petals, and an anti-reflux valve is formed. The anti-reflux valve 5 is of a triple-petal structure protruding downward. By washing, the cardia stent with one anti-reflux valve according to the present invention is obtained.

### Example 2

### A Cardia Stent with Two Anti-Reflux Valves

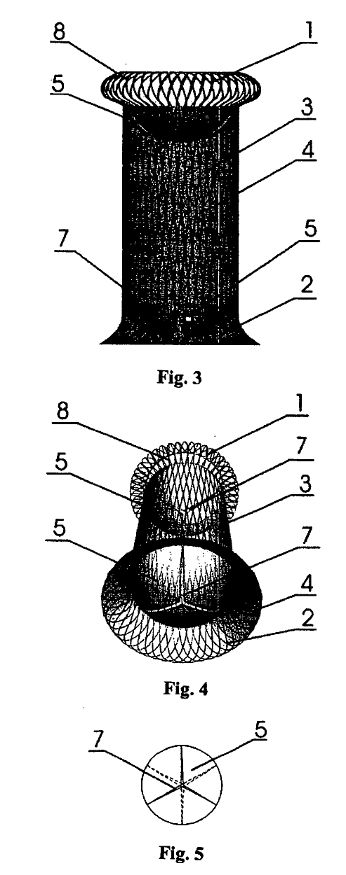

In this example, as illustrated in FIG. 3, FIG. 4 and FIG. 5, a cardia stent is made by the similar process showing in Example 1. But in this example, the cardia stent comprises two anti-reflux valves. The first anti-reflux valve is disposed at the connection part between the lower trumpet-shaped locating port 2 and the supporting net tube 3, and the second anti-reflux valve is disposed at the connection part between the upper drum-shaped locating port 1 and the supporting net tube 3. All of two anti-reflux valves 5 are of a triple-petal structure protruding downward. So the cardia stent with two anti-reflux valves according to the present invention is obtained.

### Example 3

### A Cardia Stent with One Anti-Reflux Valve and a Retrieval String

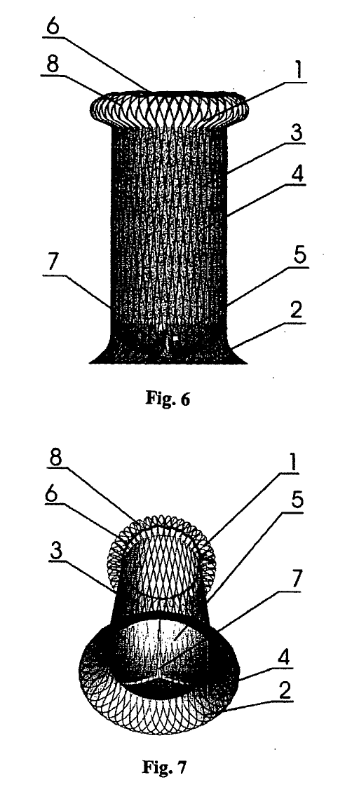

In this example, as illustrated in FIG. 6 and FIG. 7, a cardia stent is made by the similar process showing in Example 1. But in this example, we make a retrieval string (for example, surgical string) pass through the mesh of upper brim of the drum-shaped locating port 1 which is uncoated with a membrane. So the cardia stent with one anti-reflux valve and a retrieval string according to the present invention is obtained. When the retrieval string is tense, the drum-shaped locating port 1 is constringed, which cause the whole cardia stent to be constringed, then we could easily regulate the position of the cardia stent and take the whole cardia stent out of the human body.

### Example 4

### A Cardia Stent with Two Anti-Reflux Valves and a Retrieval String

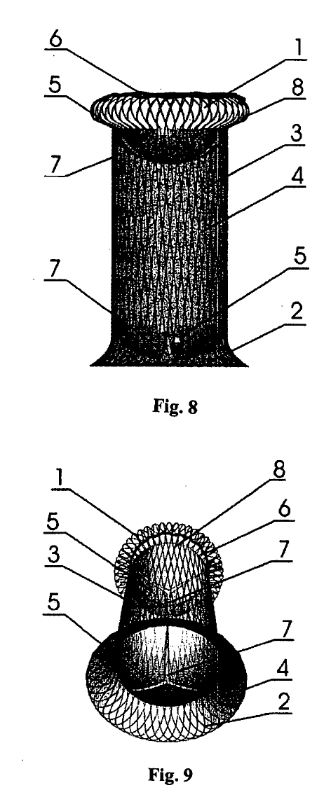

In this example, as illustrated in FIG. 8 and FIG. 9, a cardia stent is made by the similar process showing in Example 2 and Example 3. But in this example, we make a second anti-reflux valve 5. The second anti-reflux valve 5 is disposed at the connection part between the upper drum-shaped locating port 1 and the supporting net tube 3. All of two anti-reflux valves 5 are of a triple-petal structure protruding downward. So the cardia stent with two anti-reflux valves and a retrieval string according to the present invention is obtained.

In above-mentioned examples, the medical flexible material that can be implanted into a human body could be selected from the following materials: silicon rubber, Polyurethane, Polytetrafluoroethylene, and Polypropylene. And the recovery temperature and the diameter of the shape memory alloy could be adjusted according the actual situation. And the thickness and the position of the anti-reflux valve also could be adjusted according the clinical situation.

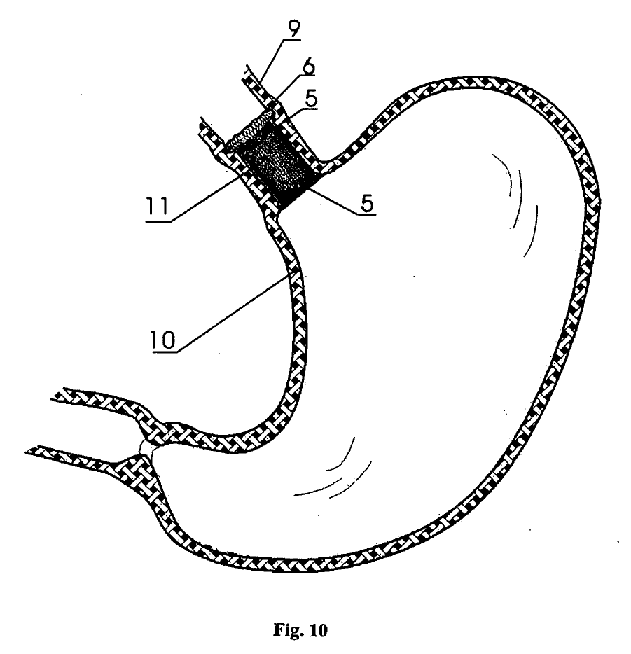

Referring to FIG. 10, when we embed the cardia stent according to the present invention, we first put the cardia stent 11 into a transporter, then let the cardia stent 11 release in the narrow position of the cardia, monitoring by the gastroscope or X-ray. The cardia stent will recover its pre-designed shape, the upper drum-shaped locating port 1 will be disposed in the oesophagus 9, and the lower trumpet-shaped locating port 2 will be disposed in the stomach 10. The anti-reflux valve 5 has the ability of resisting the corrosion of gastric juice. And the retrieval string is used to regulate the position of the cardia stent and take the whole cardia stent out of the human body easily.

What is claimed is:

1. A cardia stent used on the narrow carida, the cardia stent is characterized in:
a. said cardia stent is woven by wires (8) of shape memory NiTi alloy;
b. a drum-shaped locating port (1), disposed at the upper end of said cardia stent;
c. a trumpet-shaped locating port (2), disposed at the lower end of said cardia stent;
d. a supporting net tube (3) having a ability of resisting constringency of the cardia, connected with said upper drum-shaped locating port (1) and said lower trumpet-shaped locating port (2);
e. the remaining portions other than said drum-shaped locating port (1) are coated with a membrane (4) of medical flexible material that can be implanted into a human body; and
f. as least one anti-reflux valve (5), disposed at the connection part between said lower trumpet-shaped locating port (2) and said supporting net tube (3), or disposed at the connection part between said upper drum-shaped locating port (I) and said supporting net tube (3), or disposed at the inner part of said supporting net tube (3), wherein said anti-reflux valve (5) is of a triple-petal structure protruding downward that is made from a membrane of medical flexible material that can be implanted into a human body.

2. A cardia stent of claim 1, wherein said medical flexible material that can be implanted into a human body is selected from the following materials: silicon rubber, Polyurethane, Polytetrafluoroethylene, and Polypropylene.

3. A cardia stent of claim 1, wherein said cardia stent comprising two anti-reflux valves (5), and the vertical projection between the split of three petals of first anti-reflux valve and the split of three petals of second anti-reflux valve is not overlap.

4. A cardia stent of claim 1, wherein said cardia stent further comprising a retrieval string (6), installed in said upper drum-shaped locating port (1), which enables said upper drum-shaped locating port (1) to be constringed.

5. A cardia stent of claim 3, wherein said cardia stent further comprising a retrieval string (6), installed in said upper drum-shaped locating port (1), which enables said upper drum-shaped locating port (1) to be constringed.
